(19) United States
(12) Patent Application Publication
(10) Pub. No.: US 2026/0351424 A1
(43) Pub. Date: Nov. 5, 2026

(54) SYSTEMS AND METHODS FOR TRAINING MACHINE LEARNING SYSTEMS TO CLASSIFY SECURED DATA OBJECTS COMPRISING SETS OF INDICATORS BASED ON PREDEFINED FEATURES

(71) Applicant: AML JV, LLC, Aurora, CO (US)

(72) Inventors: Md Nazmul ISLAM, Aurora, CO (US); Clayton SMITH, Aurora, CO (US)

(73) Assignee: AML JV, LLC, Aurora, CO (US)

(21) Appl. No.: 19/661,244

(22) Filed: Apr. 28, 2026

Related U.S. Application Data
(60) Provisional application No. 63/798,372, filed on May 1, 2025.

(51) Int. Cl. G16H 50/70; G06F 18/241; G06N 20/00; G16H 10/60

(52) U.S. Cl. CPC G16H 50/70; G06F 18/241; G06N 20/00; G16H 10/60

(57) ABSTRACT

Systems and methods described herein can obtain treatment profiles for patients being treated for a disease, each profile including marker values representing a quantified state of the patient. The system can generate second treatment profiles, each corresponding to a treatment profile and including a subset of marker values. The system can configure a set of models based on the second treatment profiles to generate outcomes indicating probabilities of benefit events such as achieving an overall survival, event-free survival, etc., and risk events such as internal bleeding, etc., occurring within a predetermined time after a condition is satisfied. The system can then generate benefit-risk pairs for interventions using the models and the second treatment profiles, determine a score for the second treatment profiles based on the benefit-risk pairs, and provide data for a user interface indicating whether the therapy satisfies a threshold value based on the score.

### CROSS-REFERENCE TO RELATED APPLICATIONS

This application claims priority to U.S. Provisional Patent Application No. 63/798,372, filed May 1, 2025, which is incorporated by reference in its entirety.

### TECHNICAL FIELD

This application generally relates to techniques for training machine learning models to classify sets of indicators based on predefined features and, in some embodiments, to techniques for implementing machine learning models to determine differences across intervention types based on predefined features.

### BACKGROUND

Currently, diseases such as acute myeloid leukemia (AML) are treated in coordination with treatment plans developed by clinicians and based on standard of care therapies. These treatment plans can include the use of therapies such as administration of agents targeting specific molecules or pathways involved in the growth and survival of the disease, stem cell transplants, and/or the like. Typically, these treatments are administered in conjunction with monitoring of the response of the patient through diagnostic testing and updated to optimize a patient's outcome. But it is often difficult for clinicians to determine which therapy will be most effective in treating the targeted disease without having at least some treatment responses available to analyze. This can lead to therapies being applied to patients that are less efficient and that do not result in an optimal outcome. Additionally, it can be difficult to weigh the benefits of a particular therapy against the risks associated with that therapy in a consistent and holistic manner.

### SUMMARY

In view of the above-noted technical difficulties associated with determining whether a given therapy will be effective when treating a patient having a disease, machine learning models can be used to predict adverse reactions to therapies involving administration of an agent or agents by analyzing diverse data sources, such as electronic health records, demographic information, and chemical or biological features of drugs. These models can involve the use of advanced algorithms, including ensemble methods like random forests and deep learning techniques such as neural networks, to identify patterns in complex datasets. However, models implementing these techniques for predicting adverse drug reactions can be inefficient or imprecise due to challenges such as insufficient sample sizes, variability in patient reactions to drug dosages and durations, etc. Additionally, issues involving data heterogeneity, calibration, and limited external validation can reduce the reliability and generalizability of these models, especially when applied to rare or complex adverse reactions.

In at least some of the embodiments described herein, a system for configuring/training machine learning systems (implementing one or more models) to classify secured data objects comprising sets of indicators (e.g., correlated with marker values from treatment profiles) based on predefined features is described. The system can include one or more processors configured to execute one or more operations. These operations can cause the system obtain a set of secured data objects for individuals being treated for a condition, each secured data object comprising a set of indicator values. The system can be configured to generate a set of second secured data objects for the set of secured data objects, with each second secured data object corresponding to a secured data object and comprising a subset of indicator values from the set of indicator values. In examples, the system can be configured to train a set of machine learning systems based on the set of second secured data objects to generate outputs comprising first outputs indicating that a first event will occur and second outputs indicating that a second event will occur within a predetermined amount of time after a condition is satisfied. Each machine learning system in the set can comprise a different architecture. In examples, the system can be configured to generate a first net score for a first intervention and a second net score for a second intervention based on the set of machine learning systems and at least one second secured data object, and determine a composite score for the at least one second secured data object based on the first net score and the second net score. In examples, the system can provide data associated with a user interface to cause the user interface to be displayed on a device, the user interface indicating whether the first intervention satisfies a threshold value indicating suitability of the first intervention based on the composite score for the at least one second secured data object.

By virtue of the implementation of at least some of the aspects described herein, systems and methods can be implemented to classify treatment profiles represented as secure data objects as being correlated with patients that are or are not suitable for a given therapy or intervention to address a given condition (e.g., AML, etc.). For example, systems can be configured to process sets of indicators based on predefined features using models instead of certain statistics-based approaches that consume greater amounts of computing processor or memory when processing the underlying data. This can allow for the training and implementation of models that are both faster by virtue of the more concise amount of data being processed as well as more precise as noisy data can be identified and removed before model execution. This substantially lessens the load on the processors and decreases memory usage, allowing for efficient allocation of computing resources.

At least some of the aspects described can also improve the accuracy of the systems used to classify the secured data objects by leveraging multiple machine learning systems with different architectures, each trained on the second secured data objects. This ensemble-like approach can capture various aspects of the underlying data and produce first and second outputs that better predict when particular events will occur (e.g., the overall survival of an individual at 30 days, 60 days, etc.; the event-free survival of the individual at 30 days, 60 days, etc.; and so on). Consequently, a more reliable composite score can be generated to evaluate matching interventions. For instance, if one machine learning system excels at recognizing temporal patterns among individual indicator values, and another is more adept at recognizing temporal patterns of correlated indicators, the resulting net scores can reflect an enhanced overall accuracy in identifying which interventions are best suited based on the composite score.

Furthermore, the techniques described can conserve networking resources by reducing the volume of data transmitted across networked environments. Rather than communicating the full complement of original secured data objects, the systems described herein can share only the necessary subsets of indicator values needed for analysis. This reduction in data transmission is particularly advantageous in distributed or cloud-based machine learning scenarios, where significant bandwidth can be consumed when large volumes of data are frequently exchanged during training or when evaluating individuals at inference time. The techniques described herein can also reduce traffic congestion, decrease latency, and optimize overall network performance.

Finally, the ability of malicious third-parties to successfully launch an attack against the system can be reduced or eliminated by only exposing or sharing the second secured data objects. Since these objects contain only a subset of indicator values, adversaries can find it more challenging to reconstruct highly-sensitive data or piece together a comprehensive view of an individual's identity or health status. Moreover, employing a diverse set of machine learning architectures can further complicate infiltration attempts, as potential attackers would need to gain access to the data generated by multiple distinct models instead of a single point of vulnerability. This multi-layered structure can guard against unauthorized access and data breaches, thereby enhancing overall system security.

In one embodiment, a system for training machine learning systems to classify secured data objects comprising sets of indicators based on predefined features may comprise one or more processors; and non-transitory memory storing instructions that, when executed by the one or more processors, cause the system to: obtain a set of secured data objects for individuals being treated for a condition, each secured data object including a tokenized subject identifier and a plurality of indicator fields storing corresponding indicator values; for each secured data object: determine, for each indicator field, an association value representing correlation of the indicator field with the condition or with an intervention outcome; remove from the secured data object indicator fields having indicator values that fail a threshold criterion; encode remaining indicator values into a fixed format feature vector having fewer fields than the plurality of indicator fields; and store the fixed format feature vector in a reduced secured data object that excludes direct patient identifiers; transmit only the reduced secured data objects, and not the secured data objects, to a model-training pipeline, thereby reducing network transfer size and memory usage for model training; train, using the reduced secured data objects and a cross-validation schedule, a plurality of machine learning models having different respective architectures to generate, for a reduced secured data object and an intervention identifier, a benefit-event probability and an adverse-event probability within a predetermined time interval; exclude from subsequent scoring any machine learning model having cross-validation prediction error greater than a prediction-error threshold; execute remaining machine learning models for at least one later-obtained reduced secured data object using a first intervention identifier and a second intervention identifier to generate a first intervention score and a second intervention score from corresponding benefit-event probabilities and adverse-event probabilities; determine a composite intervention score based on a difference between the first intervention score and the second intervention score; and in response to determining that the composite intervention score satisfies a threshold, automatically generate a control record for a remote client device to modify an intervention-selection interface by enabling presentation of the first intervention and suppressing or deprioritizing presentation of the second intervention.

The instructions may further cause the system to obtain the set of secured data objects by determining one or more secured data objects that match one or more preestablished conditions from among a plurality of secured data objects; and generating the set of secured data objects based on the one or more secured data objects that match the one or more preestablished conditions.

The instructions may further cause the system to generate the reduced secured data objects by: determining p-values for each indicator field corresponding to each indicator value from the plurality of indicator fields, the p-values representing a degree of association between each indicator field and the condition; in response to comparing each p-value to a threshold p-value, determining the indicator fields having indicator values that satisfy the threshold p-value; and encoding the indicator values of the indicator fields that satisfy the threshold p-value into the fixed format feature vector of each reduced secured data object.

The threshold p-value may be 0.3 or greater.

The instructions that cause the system to train the plurality of machine learning models using the reduced secured data objects may further cause the system to: for each reduced secured data object, iteratively provide at least a portion of the fixed format feature vector as input to each machine learning model of the plurality of machine learning models until the benefit-event probability and the adverse-event probability satisfy a threshold difference when compared to a known benefit-event outcome and a known adverse-event outcome for each reduced secured data object.

The instructions may further cause the system to: remove a subset of reduced secured data objects from among the reduced secured data objects; in response to removing the subset of reduced secured data objects, train the plurality of machine learning models; and execute the plurality of machine learning models in accordance with the subset of reduced secured data objects to determine a set of prediction errors.

The subset of reduced secured data objects may comprise a first subset of reduced secured data objects, and the instructions may further cause the system to: determine that a prediction error of at least one machine learning model of the plurality of machine learning models satisfies the prediction-error threshold; remove a second subset of reduced secured data objects from among the reduced secured data objects; in response to removing the second subset of reduced secured data objects, train the plurality of machine learning models; and execute the plurality of machine learning models in accordance with the second subset of reduced secured data objects to determine a set of prediction errors.

The instructions that cause the system to execute the remaining machine learning models may further cause the system to: execute each remaining machine learning model based on the fixed format feature vector of the at least one later-obtained reduced secured data object using the first intervention identifier to generate the first intervention score and using the second intervention identifier to generate the second intervention score.

The instructions that cause the system to determine the composite intervention score may further cause the system to: determine the composite intervention score for the at least one later-obtained reduced secured data object based on a difference between benefit-event probabilities generated using the first intervention identifier and benefit-event probabilities generated using the second intervention identifier, and a difference between adverse-event probabilities generated using the first intervention identifier and adverse-event probabilities generated using the second intervention identifier, the benefit-event probabilities and adverse-event probabilities generated by executing each remaining machine learning model based on the fixed format feature vector of the at least one later-obtained reduced secured data object.

In another embodiment, a system for training machine learning systems to classify secured data objects including sets of indicators based on predefined features can include one or more processors configured to obtain a set of secured data objects for individuals being treated for a condition, each secured data object including a set of indicator values. In examples, the one or more processors can be configured to generate a set of second secured data objects for the set of secured data objects, each second secured data object corresponding to a secured data object and including a subset of indicator values from the set of indicator values. In some examples, the one or more processors can be configured to train a set of machine learning systems based on the set of second secured data objects to generate outputs including first outputs indicating that a first event will occur and second outputs indicating that a second event will occur within a predetermined amount of time after a condition is satisfied. Each machine learning system of the set of machine learning systems can include a different architecture. In examples, the one or more processors can be configured to generate a first net score for a first intervention and a second net score for a second intervention based on the set of machine learning systems and at least one second secured data object. The one or more processors can be configured to determine a composite score for the at least one second secured data object based on the first net score the second net score. And in some examples, the one or more processors can be configured to provide data associated with a user interface to cause the user interface to be displayed on a device, the user interface indicating whether the first intervention satisfies a threshold value indicating suitability of the first intervention based on the composite score for the at least one second secured data object.

The one or more processors configured to obtain the set of secured data objects can be configured to: determine one or more secured data objects that match one or more preestablished conditions from among a plurality of secured data objects; and generate the set of secured data objects on the one or more secured data objects that match the one or more preestablished conditions.

The one or more processors configured to generate the set of second secured data objects can be configured to: determine p-values for each indicator type corresponding to each indicator value from the set of indicator values, the p-values representing a degree of association between each indicator type and a condition. The one or more processors can be configured to, in response to comparing each p-value to a threshold p-value, determine the subset of indicator values based on whether each p-value satisfies the threshold p-value. The one or more processors can be configured to generate the set of second secured data objects based on the subset of indicator values. The threshold p-value can be 0.3 or greater.

The one or more processors to configure (e.g., train) the set of machine learning systems based on the set of second secured data objects can be configured to: for each secured data object of the set of second secured data objects, iteratively provide at least a portion of the subset of indicator values as input to each machine learning system of the set of machine learning systems until the first output indicating that the first event will occur and the second output indicating that the second event will occur satisfy a threshold difference when compared to a known first output indicating and a known second output indicating for each secured data object.

The one or more processors to configure the set of machine learning systems can be configured to: remove a subset of second secured data objects from among the set of second secured data objects. The one or more processors can be configured to, in response to removing the subset of secured data objects, configure the set of machine learning systems. The one or more processors can be configured to execute the set of machine learning systems in accordance with the subset of second secured data objects to determine a set of prediction errors.

The subset of second secured data objects can include a first subset of secured data objects, and the one or more processors can be further configured to: determine the prediction error of at least one machine learning system of the set of machine learning systems satisfies a prediction threshold, remove a second subset of second secured data objects from among the set of second secured data objects, and in response to removing the second subset of secured data objects, configure the set of machine learning systems. In examples, the one or more processors can be configured to execute the set of machine learning systems in accordance with the second subset of second secured data objects to determine a set of prediction errors.

The one or more processors configured to generate the first net score for the first intervention and the second net score for the second intervention based on the set of machine learning systems and the at least one secured data object can be configured to: execute each machine learning system of the set of machine learning systems based on the subset of indicator values of the at least one second secured data object to the first net score for the first intervention and the second net score for the second intervention.

The one or more processors configured to determine the composite score can be configured to: determine the composite score for the at least one second secured data object based on a difference between third outputs indicating that the first event will occur and fourth outputs indicating that the second event will occur. The third outputs and fourth outputs can be generated by executing each machine learning system of the set of machine learning systems based on the subset of indicator values of the at least one second secured data object.

In yet another embodiment, a method mat include obtaining a set of secured data objects for individuals being treated for a condition. Each secured data object can include a set of indicator values. The method can include generating a set of second secured data objects for the set of secured data objects. Each second secured data object can correspond to a secured data object and include a subset of indicator values from the set of indicator values. The method can include training a set of machine learning systems based on the set of second secured data objects to generate outputs including first outputs indicating that a first event will occur and second outputs indicating that a second event will occur within a predetermined amount of time after a condition is satisfied. Each machine learning system of the set of machine learning systems can include a different architecture. The method can include generating a first net score for a first intervention and a second net score for a second intervention based on the set of machine learning systems and at least one second secured data object. In examples, the method can include determining a composite score for the at least one second secured data object based on the first net score the second net score. The method can include providing data associated with a user interface to cause the user interface to be displayed on a device, the user interface indicating whether the first intervention satisfies a threshold value indicating suitability of the first intervention based on the composite score for the at least one second secured data object.

Obtaining the set of secured data objects can include determining one or more secured data objects that match one or more preestablished conditions from among a plurality of secured data objects, and generating the set of secured data objects on the one or more secured data objects that match the one or more preestablished conditions.

Generating the set of second secured data objects can include determining p-values for each indicator type corresponding to each indicator value from the set of indicator values, the p-values representing a degree of association between each indicator type and a condition; in response to comparing each p-value to a threshold p-value, determining the subset of indicator values based on whether each p-value satisfies the threshold p-value; and generating the set of second secured data objects based on the subset of indicator values. In some aspects, the threshold p-value can be 0.3 or greater.

Configuring the set of machine learning systems based on the set of second secured data objects can include, for each secured data object of the set of second secured data objects, iteratively providing at least a portion of the subset of indicator values as input to each machine learning system of the set of machine learning systems until the first output indicating that the first event will occur and the second output indicating that the second event will occur satisfy a threshold difference when compared to a known first output indicating and a known second output indicating for each secured data object.

Configuring the set of machine learning systems can include removing a subset of second secured data objects from among the set of second secured data objects. The method can include, in response to removing the subset of secured data objects, configuring the set of machine learning systems. In examples, the method can include executing the set of machine learning systems in accordance with the subset of second secured data objects to determine a set of prediction errors.

The subset of second secured data objects includes a first subset of secured data objects. In these aspects, the method can further include determining the prediction error of at least one machine learning system of the set of machine learning systems satisfies a prediction threshold, removing a second subset of one or more second secured data objects from among the set of second secured data objects, and in response to removing the second subset of secured data objects, configuring the set of machine learning systems. In examples, the method can include executing the set of machine learning systems in accordance with the second subset of second secured data objects to determine a set of prediction errors.

Generating the first net score for the first intervention and the second net score for the second intervention based on the set of machine learning systems and the at least one secured data object can include executing each machine learning system of the set of machine learning systems based on the subset of indicator values of the at least one second secured data object to the first net score for the first intervention and the second net score for the second intervention.

Determining the composite score can include determining the composite score for the at least one second secured data object based on a difference between third outputs indicating that the first event will occur and fourth outputs indicating that the second event will occur. The third outputs and fourth outputs can be generated by executing each machine learning system of the set of machine learning systems based on the subset of indicator values of the at least one second secured data object.

In another embodiment, one or more non-transitory computer-readable mediums storing instructions thereon, when executed by one or more processors, can cause the one or more processors obtain a set of secured data objects for individuals being treated for a condition, each secured data object including a set of indicator values. The instructions can cause the one or more processors to generate a set of second secured data objects for the set of secured data objects, each second secured data object corresponding to a secured data object and including a subset of indicator values from the set of indicator values. The instructions can cause the one or more processors to train a set of machine learning systems based on the set of second secured data objects to generate outputs including first outputs indicating that a first event will occur and second outputs indicating that a second event will occur within a predetermined amount of time after a condition is satisfied. Each machine learning system of the set of machine learning systems can include a different architecture. The instructions can cause the one or more processors to generate a first net score for a first intervention and a second net score for a second intervention based on the set of machine learning systems and at least one second secured data object. The instructions can cause the one or more processors to determine a composite score for the at least one second secured data object based on the first net score the second net score. The instructions can cause the one or more processors to provide data associated with a user interface to cause the user interface to be displayed on a device, the user interface indicating whether the first intervention satisfies a threshold value indicating suitability of the first intervention based on the composite score for the at least one second secured data object

The instructions that cause the one or more processors to obtain the set of secured data objects can cause the one or more processors to determine one or more secured data objects that match one or more preestablished conditions from among a plurality of secured data objects, and generate the set of secured data objects on the one or more secured data objects that match the one or more preestablished conditions.

It is to be understood that both the foregoing general description and the following detailed description are exemplary and explanatory and are intended to provide further explanation of the embodiments described herein.

### BRIEF DESCRIPTION OF THE DRAWINGS

The accompanying drawings constitute a part of this specification, illustrate one or more embodiments and, together with the specification, explain the subject matter of the disclosure.

FIG. 1 is a block diagram of an environment for training machine learning models to classify sets of indicators based on predefined features, in accordance with one or more embodiments described herein.

FIG. 2 is a flow diagram illustrating operations of a method for training machine learning models to classify sets of indicators based on predefined features, in accordance with one or more embodiments described herein.

FIGS. 3A-3D are a diagram illustrating a process for analyzing data objects using machine learning models described with respect to FIG. 2, in accordance with one or more embodiments described herein.

FIG. 4 is a diagram illustrating an interactive user interface that can be implemented, in accordance with one or more embodiments described herein.

FIG. 5 is a flow chart illustrating a process for reducing secured data objects by removing indicator fields that fail a threshold criterion, encoding remaining indicator values into fixed format feature vectors, training a plurality of machine learning models, excluding models that fail a prediction-error threshold, generating intervention scores, determining a composite intervention score, and modifying an intervention-selection interface at a remote client device, in accordance with one or more embodiments described herein.

### DETAILED DESCRIPTION

Reference will now be made to the embodiments illustrated in the drawings, and specific language will be used here to describe the same. It will nevertheless be understood that no limitation of the scope of the disclosure is thereby intended. Alterations and further modifications of the features illustrated here, and additional applications of the principles as illustrated here, which would occur to a person skilled in the relevant art and having possession of this disclosure, are to be considered within the scope of the disclosure.

The present disclosure involves selecting a subset of treatment profiles obtained for a plurality of patients (treated for a specific disease or controls) and training a set of machine learning-based models (referred to generally as a “set of models”) to predict probabilities that various benefits/risks (e.g., overall survival, event-free survival, etc., at 30, 60, 90 days; febrile neutropenia, creatinine, anemia, etc., at 30, 60, 90 days) will occur. Once trained, the set of models can then be executed on a per-patient basis to determine benefit/risk scores associated with administration of a therapy for each patient. These scores can then be used to determine a difference of differences which represents a score attributed to a benefit (e.g., event-free survival if venetoclax (ven) and azacitidine (aza) are administered, alone or in any combination with chemotherapy (cusa)) and its corresponding risks, when compared to the benefit of other therapies (e.g., a different treatment, not administering ven/aza/cusa, etc.). This score can then be used to determine a degree of benefit when compared to corresponding risks (e.g., low, neutral, high) and inform clinicians on whether or not a particular therapy for a particular patient will be, on balance, effective in treating an underlying disease. While the present disclosure is described with respect to the administration of certain therapies to treat AML (e.g., ven/aza/cusa), it will be understood that the techniques described can be applied to address any suitable disease/therapy combination.

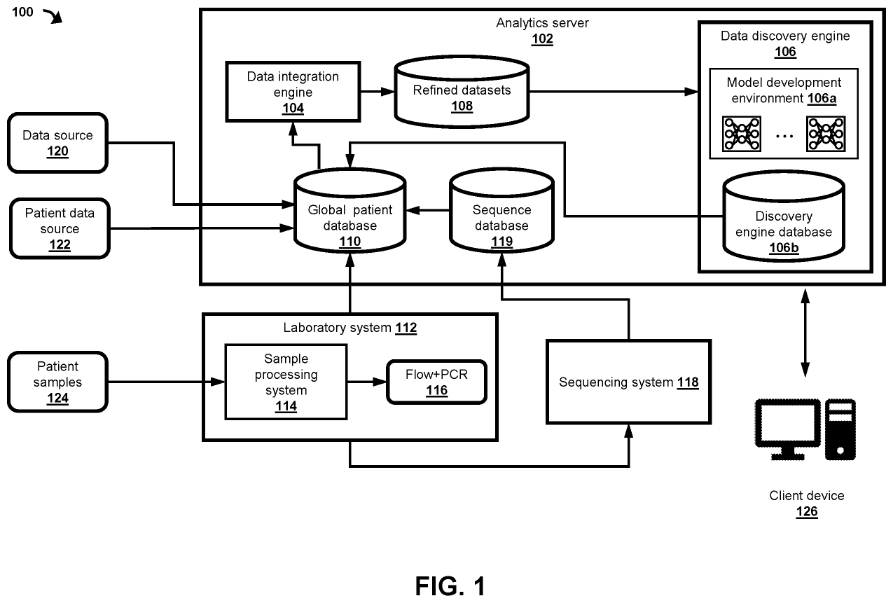

FIG. 1 is a block diagram of an environment 100 for training machine learning models to classify sets of indicators based on predefined features, according to an embodiment. The environment 100 can include an analytics server 102, a laboratory system 112, a sequencing system 118, a data source 120, patient data source 122, patient samples 124, and a client device 126. Various components depicted in FIG. 1 can belong to an organization involved in clinical research of one or more diseases such as, for example, acute myeloid leukemia (AML) or other diseases and/or to one or more organizations involved in treating patients with the one or more diseases. While certain components and devices are illustrated as being included in the environment 100 of FIG. 1, it will be understood that the environment 100 is not confined to the components or diseases as described herein and can include additional or different components (not shown for purposes of brevity and clarity) which are configured to be considered within the scope of the embodiments described herein.

In some embodiments, the analytics server 102 can include any computing device comprising a processor and non-transitory machine-readable storage capable of executing the various tasks, processes, and/or operations as described herein. The analytics server 102 can employ various processors such as central processing units (CPUs), graphical processing units (GPUs), and/or the like. Some non-limiting examples of such computing devices can include workstation computers, laptop computers, server computers, and/or the like. While the environment 100 includes a single analytics server 102, there can be multiple analytics servers 102. Further, the analytics server 102 can include any number of computing devices operating in a distributed computing environment such as, for example, a cloud computing environment. As described herein, the analytics server 102 can include a data integration engine 104, a data discovery engine 106, refined datasets 108, a global patient database 110, and a sequence database 119. In some embodiments, the analytics server 102 can include and/or implement operations that are associated with the laboratory system 112, the sequencing system 118, and/or the client device 126. In some embodiments, the analytics server 102 can include and/or implement operations that are associated with (e.g., involved in the generation of) the data source 120, the patient data source 122, and/or the patient samples 124.

In some embodiments, the analytics server 102 can be configured to receive data from the data source 120, the patient data source 122, and the laboratory system 112 and sequencing system 118 when processing patient samples 124. For example, the analytics server 102 can be configured to receive data from the data source 120, where the data is associated with (e.g., represents) entries corresponding to one or more patient files. As an example, as patients interact with clinicians, the clinicians can generate information that are received as input at a client device (not explicitly illustrated) that is associated with the clinicians, the notes indicating clinical observations and/or updates to treatment plans for the patients made by the clinician. The client device can then generate patient data that is associated with each patient and representative of the clinical observations or updates to the treatment plans and store the patient data in the data source 120 to later transmit to the analytics server 102. In this example, the analytics server 102 can implement the global patient database 110 such that the patient data is uploaded and stored in the global patient database 110 in association with one or more identifiers for the patient as described herein.

In another example, the analytics server 102 can be configured to receive data from the patient data source 122, where the data is associated with (e.g., represents) information about individual patients. As an example, as a history of a patient is obtained, the clinicians and/or the patients can generate information that is received as input at a client device (not explicitly illustrated) that is associated with the clinicians and/or patients, the information indicating aspects of the history of the patient such as whether the patient is associated with a history of a given disease in their family, whether the patient had any exposure to environmental conditions associated with the given disease, and/or the like. The client device can then generate patient data that is associated with each patient and representative of the history of the patient and store the patient data in the patient data source 122 to later transmit to the analytics server 102. In this example, the analytics server 102 can obtain and store the patient data in the global patient database 110 in association with one or more identifiers for the patient as described herein.

In yet another example, the analytics server 102 can be configured to receive data from the laboratory system 112 and/or the sequencing system 118, where the data is associated with (e.g., represents) information about patient samples (e.g., tissue samples, blood samples, blood counts (e.g., complete blood counts), bone marrow aspiration and biopsy results, lumbar puncture results, and/or the like) as well as the results of the processing of the samples (e.g., a DNA sequence or targets thereof). As an example, as a patient is evaluated and/or treated for a disease such as AML, patient samples 124 similar to those described above can be obtained. The patient samples 124 can be initially obtained and processed by a laboratory system 112 and processed by a sample processing system 114. The sample processing system 114 can implement one or more devices configured to obtain and store the patient samples and extract DNA from the patient samples. For example, in preparation for genetic analysis to guide AML treatment, patient blood or bone marrow can first be obtained from a patient and frozen. Later, these samples can be quality checked to ensure the sample purity and quantity are sufficient for sequencing. In some embodiments, the isolated DNA can then undergo further processing to be separated into manageable fragments and equipped with adapters (e.g., short, specific pieces of synthetic DNA associated with the fragmented DNA molecules) for compatibility with sequencing machines. In some embodiments, the samples can also be provided to a flow and polymerase chain reaction (PCR) system to extract and amplify the isolated DNA. The laboratory system 112 can then provide the processed samples and corresponding data representing the samples to be processed by the sequencing system 118. Additionally, or alternatively, the laboratory system 112 can then provide the data generated by the laboratory system 112 when processing the samples to the analytics server 102 to be stored in the global patient database 110.

In some embodiments, the sequencing system 118 can be configured to receive the patient samples and/or the isolated DNA and sequence the patient samples. In one example, the sequencing system 118 can attach DNA fragments to a surface in a specific pattern, creating clusters. The sequencing itself can involve a series of cycles where fluorescently labeled nucleotides are introduced one by one. The incorporation of each base can be detected, identifying the sequence of the fragment base by base. Finally, the sequencing system 118 can analyze the vast amount of data, assemble the original DNA sequences, and identify any variations or mutations present (sometimes referred to as Next-Generation Sequencing (NGS)). The sequencing system 118 can then provide data associated with the sequenced DNA to the analytics server 102. In this example, the analytics server 102 can store the sequenced DNA in a sequence database 119 that stores the sequenced DNA in association with one or more patient identifiers established by the analytics server 102. In some embodiments, the analytics server 102 can also cause the sequence database 119 to provide the data associated with the sequenced DNA to the global patient database 110 to be stored in association with other data associated with the patient such as a treatment profile and/or limited treatment profile for the patient as described herein.

In some embodiments, the analytics server 102 can implement a data integration engine 104 to process data stored in the global patient database 110. For example, the analytics server 102 can implement the data integration engine 104 such that the data integration engine 104 is configured to obtain the data associated with the patients that is stored in the global patient database 110 and processes the data to be used by the data discovery engine 106. In one example, as data is obtained by the global patient database 110 for a given patient, the data can be stored in the global patient database 110 in association with one or more identifiers as part of a profile for the patient. The data integration engine 104 can then obtain the data associated with the patient (e.g., the entire profile or portions thereof) from the global patient database 110 and process the data to generate a limited treatment profile. The limited treatment profile can then be stored in the refined datasets database 108 (referred to herein as “refined datasets”) and made available to the data discovery engine 106. In this way, the analytics server 102 can maintain two separate datasets that allow for updates to the limited treatment profiles stored in the refined datasets 108 and subsequent use by the data discovery engine 106 when performing the operations described herein. As will be understood, in this example the data associated with the patient that is stored in the global patient database 110 can be updated over time such that the patient profile is represented as a set of entries associated with a time series. As the global patient database 110 is updated, the data integration engine 104 can obtain updated versions of the data associated with the patient from the global patient database 110, process the data when updating the limited treatment profiles in the refined datasets 108, and store the updates in the refined datasets 108.

In some embodiments, the analytics server 102 can implement the data discovery engine 106 that includes a model development environment 106a and a discovery engine database 106b. For example, the analytics server 102 can implement the data discovery engine 106 such that the data discovery engine 106 is configured to receive data associated with one or more limited treatment profiles that are stored in the refined datasets 108 and process the one or more limited treatment profiles. In this example, the analytics server 102 can process the one or more limited treatment profiles using the model development environment 106a. Processing the limited treatment profiles can include providing the limited treatment profiles to one or more models (e.g., machine learning-based models and/or the like) to determine one or more metrics (or performance metrics) corresponding to the performance of each of the models to indicate which model is most accurate, efficient, and/or the like at generating one or more predictions. These predictions can include indications of treatment options that have a likelihood of optimizing an outcome (e.g., lifespan) for the patients. Processing the limited treatment profiles can additionally, or alternatively, include determining one or more aspects of the limited treatment profiles. For example, where the limited treatment profiles is associated with a predetermined number of possible attributes but the patient samples 124 obtained to be processed were limited and only usable to determine a subset of the possible attributes, the model development environment 106a can process the portions of the refined patient profile that are available in the refined datasets 108 to determine one or more of the remaining attributes. In this example, data associated with the one or more remaining attributes can be stored by the data discovery engine 106 in the discovery engine database 106b. The analytics server 102 can then periodically or in real-time update the global patient database 110 based on the data associated with the limited treatment profiles (e.g., the one or more remaining attributes and/or the like) that are stored in the discovery engine database 106b.

In some embodiments, the client device 126 can include any computing device comprising a processor and non-transitory machine-readable storage capable of executing the various tasks, processes, and/or operations as described herein. The client device 126 can employ various processors such as central processing units (CPUs), graphical processing units (GPUs), and/or the like. Some non-limiting examples of such computing devices can include workstation computers, laptop computers, server computers, and/or the like. While the environment 100 includes a single client device 126, there can be multiple client devices 126. Further, the client device 126 can include any number of computing devices operating in a distributed computing environment such as, for example, a cloud computing environment. In some embodiments, the client device 126 can be associated with one or more software developers and/or one or more clinicians that are interacting with (e.g., configuring operation of) the analytics server 102 as described herein. In some embodiments, the client device 126 can be associated with one or more clinicians and/or one or more organizations involved in treating patients with the one or more diseases such as a hospital and/or the like.

In some embodiments, the analytics server 102 can generate and display an electronic platform (e.g., via the client device 126) when receiving and processing patient data associated with one or more patients, performing one or more operations when analyzing the patient data, and outputting data associated with the results of the operations performed by any of the components of the analytics server 102 such as, for example, the data discovery engine 106. The electronic platform can include graphical user interfaces (GUI) displayed by display devices of one or more client devices 126. An example of the electronic platform generated and hosted by the analytics server 102 can be a web-based application or a website configured to be displayed on different electronic devices, such as mobile devices, tablets, personal computers, and the like.

The above-mentioned components can be configured to interconnect with to each other and establish communication connections therebetween through a network (not explicitly illustrated). Examples of the network can include, but are not limited to, private or public local-area-networks (LAN), wireless LAN (WLAN) networks, metropolitan area networks (MAN), wide-area networks (WAN), and the Internet. The network can include wired and/or wireless communications according to one or more standards and/or via one or more transport mediums. The communication over the network can be performed in accordance with various communication protocols such as Transmission Control Protocol and Internet Protocol (TCP/IP), User Datagram Protocol (UDP), and IEEE communication protocols. In one example, the network can include wireless communications according to Bluetooth specification sets or another standard or proprietary wireless communication protocol. In another example, the network can also include communications over a cellular network, including, e.g., a GSM (Global System for Mobile Communications), CDMA (Code Division Multiple Access), and EDGE (Enhanced Data for Global Evolution) network.

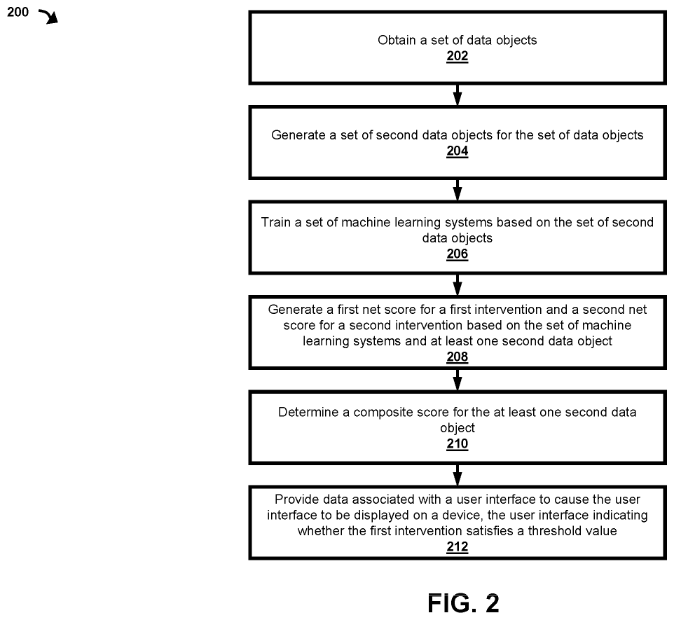

FIG. 2 is a flow diagram illustrating operations of a method 200 for training machine learning models to classify sets of marker values based on predefined features, in accordance with one or more embodiments described herein. In some implementations, one or more of the functions described with respect to the method 200 can be performed (e.g., completely, partially, and/or the like) by an analytics server that is the same as, or similar to, the analytics server 102 of FIG. 1. In some implementations, one or more of the functions described with respect to the method 200 can be performed (e.g., completely, partially, and/or the like) by another device or group of devices separate from and/or including the analytics server, such as by one or more client devices that are the same as, or similar to, the client device 126 of FIG. 1.

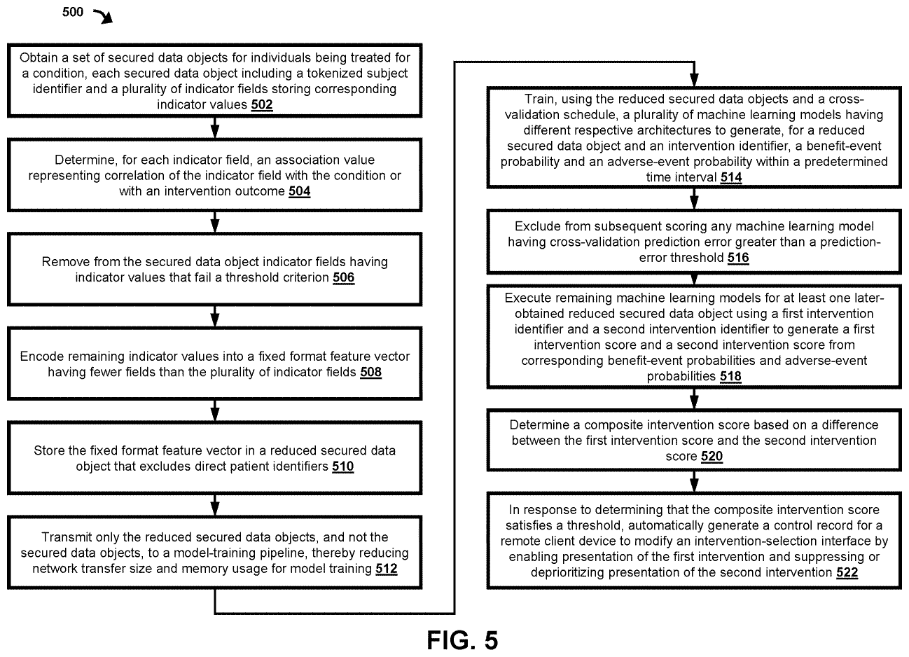

At operation 202, the analytics server can obtain a set of data objects (e.g., secured data objects such as entries in a dataset correlated with and/or representing treatment profiles established for patients). For example, the analytics server can obtain a set of data objects that are correlated with individuals either being treated for a condition (e.g., a disease such as AML) or not treated for a condition (e.g., individuals with the condition who have not been treated representing controls). In examples, each data object can include a set of indicator values (e.g., biomarker values) that represent the state of a given individual. For example, the analytics server can obtain the set of data objects, where the data objects are associated with entries in a dataset (e.g., maintained in a global patient database and/or a refined dataset as described herein). Each entry can correspond to a treatment profile of an individual that is diagnosed as having or not having a disease/condition (e.g., AML, etc.). In some implementations, each secured data object can include a tokenized subject identifier and a plurality of indicator fields storing corresponding indicator values, as described with respect to operation 502 of FIG. 5. The tokenized subject identifier can represent a de-identified reference to the individual, and the plurality of indicator fields can store quantitative measurements associated with the condition and the treatment of the individual. While the techniques discussed refer to diseases such as AML, it will be understood that they are not limited to AML and that they can be implemented in view of any suitable combination of conditions/diseases. Additionally, while reference throughout will be made to conditions and/or diseases where contextually appropriate to maintain clarity, the techniques of the present disclosure are not limited to the examples described unless explicitly stated otherwise.

In some embodiments, each data object can include indicators (e.g., biomarkers, etc.) represented by one or more marker values measured at points in time over a period of time. For example, each marker value can include a value that provides a quantitative measurement of a physiological or molecular state of the individual represented by the treatment profile at a point in time. In an example, each data object can be generated and/or updated to include indicators that are recorded at a point in time when an individual is suspected of having a particular disease or has been diagnosed with the disease and is progressing through treatment. Additionally, each treatment profile can include information about the individual (e.g., their age, environmental conditions, reported symptoms, etc.) when the biomarkers are measured. As a result, each data object can represent a treatment profile that correlates quantified measurements representing the state of the patient through a progression of a disease over time as well as ways in which a particular disease(s)/condition(s) are treated. In examples, the marker values and/or information described can be determined based on a laboratory system (as described above) generating biomarker data associated with the marker values described herein. Additionally, or alternatively, the marker values and/or information can be determined based on a sequencing system generating sequence data associated with one or more nucleotide sequences including raw reads, aligned reads, variant data, expression data, etc. And in some examples, the marker values can be determined based on inputs received by a clinician operating the client device when observing the treatment over a period of time. This biomarker data can then be used to update the respective data objects to create a time series representation of the state of the patient when monitored.

In some embodiments, the analytics server can be configured to obtain the set of data objects based on analyzing a dataset including a plurality of data objects and determining that each data object of the set of data objects matches one or more preestablished conditions. For example, the analytics server can evaluate one or more marker values established by treatment profiles correlated with the plurality of data objects in the dataset. This can involve evaluating the marker values (individually or in predetermined combinations) to identify data objects in the set of data objects with marker values and/or combinations of marker values within predetermined ranges indicating a disease. Additionally, or alternatively, the analytics server can be configured to filter the plurality of data objects to identify data objects where patients are being monitored for the disease because of particular events (e.g., reporting symptoms or combinations of symptoms associated with AML), etc. In some examples, the analytics server can compare the marker values to threshold-based conditions to identify the data objects that are relevant (e.g., indicative of patients possibly having AML). In at least some examples, the analytics server can then generate the set of data objects by extracting the one or more data objects matching the one or more preestablished conditions from the dataset. For example, the analytics server can generate the set of data objects and store the set of data objects for use when executing subsequent operations to train/configure machine learning systems to classify subsequently-obtained data objects as described herein.

In some embodiments, to obtain the set of data objects, the analytics server can analyze the indicators of the data objects in the dataset (where the plurality of data objects are stored) associated with patients known to have a disease/condition (e.g., identified by the analytics server as having marker values that correspond to known marker value ranges indicative of the disease/condition) and determine, for each indicator field of each data object, an association value representing a correlation of the indicator field with the condition or with an intervention outcome, as described with respect to operation 504 of FIG. 5, where the association value indicates a degree of correlation between the indicators and the disease. For example, the analytics server can determine p-values for each indicator type in the plurality of data objects. In some examples, the analytics server can compute the p-values representing a degree of association between each indicator type and a condition (that the individual represented by a given data object is diagnosed with AML or is expected to be diagnosed with AML). In some implementations, the analytics server can remove from each secured data object indicator fields having indicator values that fail a threshold criterion, as described with respect to operation 506 of FIG. 5. After determining the p-values for each indicator type, the analytics server can compare each p-value to a threshold p-value (e.g., a p-value set by a clinician directly or indirectly controlling the analytics server) and determine the subset of indicator types having indicator values that satisfy the threshold p-value. In some examples, the analytics server can then generate the set of second data objects by extracting the indicators from the data objects in the dataset where the indicators are identified as satisfying the threshold p-value. In examples, the p-value can be 0.2, 0.3, etc.

At operation 204, the analytics server can generate a set of second data objects for the set of data objects. For example, the analytics server can generate the set of second data objects, where each second data object corresponds to a data object from the set of data objects and includes a subset of the indicator values therefrom (e.g., indicators with p-values that satisfy the above-noted threshold p-value). In some embodiments, the analytics server can process each data object to extract one or more indicator values correlated with the disease or condition being analyzed and generate each second data object comprising a subset of these indicator values. For example, the analytics server can select a subset of the indicator values from the set of indicator values by applying predetermined criteria (e.g., a selection rule such as only including indicator values correlated with indicators that have a p-value that satisfies the threshold p-value, etc.) to identify the indicator values that are correlated with a disease such as AML (alone or in combination with other indicator values). In some implementations, the analytics server can encode remaining indicator values into a fixed format feature vector having fewer fields than the plurality of indicator fields, as described with respect to operation 508 of FIG. 5. The analytics server can store the fixed format feature vector in a reduced secured data object that excludes direct patient identifiers, as described with respect to operation 510 of FIG. 5. In this example, the analytics server can then generate the second data objects to be used to train a set of machine learning models (described as implemented by machine learning systems), where each indicator type represented by the second data objects corresponds to a feature used by the machine learning models during training/inference. In some implementations, the analytics server can transmit the reduced secured data objects, and not the original secured data objects, to a model-training pipeline, thereby reducing network transfer size and memory usage for model training, as described with respect to operation 512 of FIG. 5.

In some embodiments, the analytics server can be configured to filter the plurality of data objects based on one or more criteria when determining the set of data objects. For example, the analytics server can filter the first plurality of data objects based on criteria indicating administration of an agent or collection of agents (e.g., venetoclax (ven), azacitidine (aza), and/or cytarabine-based chemotherapy (cusa)) to the individuals represented by the dataset. In this example, the analytics server can be configured to filter the first plurality of data objects based on whether the individuals corresponding to each data object were treated using the agent or collection of agents to adjust progression of the disease. In some embodiments, the analytics server can filter the first plurality of data objects based on one or more other criteria. For example, the analytics server can be configured to filter the first plurality of data objects based on an age of the individuals represented by the first plurality of data objects. In these examples, the analytics server can then determine the second data objects based on filtering the plurality of data objects. While the present disclosure discusses filtering the first plurality of data objects in accordance with a targeted agent or collection of agents used to treat AML, it will be understood that any suitable filter can be applied.

At operation 206, the analytics server can train a set of machine learning systems based on the set of second data objects. For example, the analytics server can train a set of machine learning systems (e.g., each implementing corresponding machine learning models or combinations/ensembles of machine learning models) based on the set of second data objects to generate outputs indicating that one or more events will occur. In an example, the analytics server can train the set of machine learning systems to generate an output indicating that the one or more events will occur within a predetermined amount of time after a condition is satisfied (e.g., after indicators are determined to satisfy one or more conditions indicative of a disease such as AML, after a therapy is administered, etc.). In some examples, each machine learning system of the set of machine learning systems can include a different architecture (e.g., a convolutional neural network, a decision tree system, a support vector machine, etc.) to allow a diverse set of approaches in generating the outputs described herein. In this example, training can comprise processing the second secured data objects using each machine learning system so as to allow the outputs to correspond to their respective conditions and events. In some implementations, the analytics server can train the set of machine learning systems using the reduced secured data objects and a cross-validation schedule, as described with respect to operation 514 of FIG. 5, where each machine learning model of a plurality of machine learning models has a different respective architecture and generates, for a reduced secured data object and an intervention identifier, a benefit-event probability and an adverse-event probability within a predetermined time interval.

In some embodiments, the first output can represent one or more probabilities that one or more first events occur. These first events can be classified as benefits, such as reaching an overall survival date, reaching an event-free survival date, reaching a relapse free survival date, satisfying a response duration, having a particular treatment response (represented by marker values reaching and/or remaining in a particular range), and/or having a best response (for example, complete response or complete response with incomplete blood count recovery during a first treatment cycle). As an example, in response to the analytics server configuring the set of machine learning systems to predict overall survival at 30 days after treatment for AML is initiated (e.g., after ven/aza or ven/aza/cusa is administered), for each data object provided as an input to the machine learning systems, first outputs can be generated indicating probabilities that individuals correlated with data objects processed by the machine learning systems will survive to 30 days after treatment. In this context, the first outputs can correspond to benefit-event probabilities as described with respect to operation 518 of FIG. 5.

Similarly, the second output can represent probabilities that one or more second events occur. These second events can be classified as risks associated with the administration of a therapy (e.g., ven/aza, ven/aza/cusa, etc.) or not administering the therapy. For example, the second events can include the individual experiencing febrile neutropenia, creatinine, anemia, neutrophils, bilirubin, etc. As an example, in response to the analytics server configuring the set of machine learning systems to predict that individuals correlated with data objects will experience febrile neutropenia after treatment for AML is initiated, the machine learning systems can be configured to generate outputs indicating probabilities that individuals correlated with data objects processed by the machine learning systems will experience febrile neutropenia at 30 days after initiating treatment for AML. In this context, the second outputs can correspond to adverse-event probabilities as described with respect to operation 518 of FIG. 5.

In some embodiments, the analytics server can select one or more machine learning systems to configure/train to generate outputs as described above. For example, the analytics server can select and implement machine learning systems that incorporate a single machine learning model or an ensemble of models that have different model architectures to receive data objects as inputs and generate outputs indicating probabilities that one or more events will occur. In some examples, the architectures can include convolutional neural networks, multilayer perceptrons, recurrent neural networks, decision tree ensembles, etc. In at least some of the examples described, ensemble architectures can combine multiple models in parallel or sequential configurations. For example, the analytics server can implement an ensemble architecture where each model is implemented in parallel and the corresponding probabilities output by the models are averaged to determine an average probability for a given event. In another example, the analytics server can configure one or more models to interoperate to generate a single output indicative of the probability that a given event will occur.

In some embodiments, the analytics server can configure the model(s) to receive inputs such as data objects having marker values collected at predetermined instances during a treatment process. For example, the inputs can be derived from information represented by the data object, such as patient records, sensor data, or other clinical measurements and can include marker values, treatment parameters, and timestamps associated with treatment events such as a predetermined intervention like a stem cell transplant. The analytics server can then implement the models to produce outputs that include probabilities that one or more events will occur at points in time after treatment is administered. In other examples, the analytics server can implement the models to produce outputs as probability distributions or vectors that can indicate the likelihood of events occurring at various future points in time (e.g., probabilities for overall survival, event-free survival, etc., at 30 days, 60 days, etc.).

In some embodiments, the analytics server can train the models described herein using supervised learning techniques. For example, the analytics server can provide a data object (e.g., a second data object having a subset of all available features that are compatible with the model being trained) as an input (also referred to as a “training input”) to the models to cause the models to generate outputs. More specifically, the inputs can include marker values and indications of whether a particular therapy was administered, and the outputs can include probability values that represent the occurrence of events (e.g., benefit events or risk events) at particular times after treatment is administered. The analytics server can then iteratively update the weights of the models (e.g., by implementing backpropagation techniques, etc.) by comparing the predicted probability values with the actual event occurrence data associated with annotations of the data objects that one or more events occurred (e.g., the individual lived to a particular overall survival date, etc.) represented by the data object and then adjusting the weights of the models based on a difference between the predicted probability values and the actual event occurrence data. The analytics server repeat this process and update the weights of the models across training epochs until a desired difference (e.g., a threshold difference) is satisfied indicating the models have converged.

In some embodiments, the analytics server can train the models as described above iteratively using a leave-one-out cross validation (LOOCV) technique. For example, the analytics server can partition a set of second data objects such that one or more second data objects are reserved for use when evaluating the performance of the model while the remaining second data objects are used as training data. The analytics server can then train/configure the models using the remaining second data objects to generate predictions that one or more events will occur as described herein and update the weights across epochs until model performance converges. In some examples, iterative training cycles can be performed by changeably reserving/removing each second data object for evaluation at each epoch, which can allow the system to compute performance metrics (e.g., prediction errors) for each reserved data object and then adjust the model accordingly. In some examples, the analytics server can implement the LOOCV technique to assess the ability of the model to predict events across different subsets of training data and can provide a framework for iterative refinement of the parameters (weights) of the model.

At operation 208, the analytics server can generate a first net score for a first intervention and a second net score for a second intervention based on (e.g. using) the set of machine learning systems and at least one second data object (also referred to as a later-obtained data object when used at inference time). For example, after training the machine learning models implemented by the machine learning systems, the analytics server can obtain (e.g., receive) a later-obtained data object for evaluation from a client device. The client device can be controlled by a clinician treating a patient with a condition/disease that the model is configured for, such as AML. In this example, the analytics server can be configured to provide the later-obtained data object as an input twice, first with an indication of the administration of a therapy and second with an indication that a different therapy (or no therapy) is administered). This can cause the machine learning systems to generate outputs indicating probabilities that various events will occur, allowing the analytics server to determine a first net score for a first intervention (e.g., administration of ven/aza or ven/aza/cusa to treat AML) and a second net score for a second intervention (e.g., administration of the other of ven/aza or ven/aza/cusa or non-administration of any agent).

To determine the first net score and the second net score described above, the analytics server can provide data objects, such as the later-obtained data object, as an input to the machine learning systems along with an indication of whether one or more therapies were administered to cause the machine learning systems to generate respective outputs representing scores (e.g., a probability) that one or more first events (e.g., benefit events) and one or more second events (e.g., risk events) will occur. The analytics server can then determine the first net score representing probabilities that certain events (benefit events and risk events) will occur after administration of a therapy based on the score for the one or more first events and the one or more second events attributable to administration of a first therapy, and a second net score based on the score representing probabilities that certain events will occur after administration of a different therapy. The analytics server can aggregate (e.g., add) the scores generated for a given data object and therapy for the one or more first events (benefit events) and subtract the scores of the one or more second events (e.g., risk events) to determine a net score. Additionally, or alternatively, the analytics server can identify pairs of first events and second events and generate net scores for the pairs, respectively. In examples, the analytics server can use the outputs from the machine learning systems to determine the first net score and second net score to allow for comparison of outcomes associated with the first intervention with any number of different interventions.

At operation 210, the analytics server can determine a composite score for the at least one second data object based on the first net score the second net score. For example, the analytics server can determine a composite score for at least one second data object based on the first net score and the second net score. In some examples, the composite score can be represented as a difference of differences (illustrated below), where the probability that a first event (for example, a benefit) occurs and the probability that a second event (for example, a risk) occurs as a result of a first and second intervention are each computed and compared to determine the composite score. In an example, the analytics server can determine the composite score by computing a difference between the probability values associated with the first net score and the second net score. This can include determining each pair of probabilities and then aggregating the computed differences to use an overall composite score. In some examples, the analytics server can combine computed differential values across multiple event pairs (for example, using a summation or other arithmetic operation) to allow a comprehensive evaluation of the later-received data object. In this example, the composite score can allow further analysis by providing an indicator that can be compared across different second data objects for subsequent processing.

To determine the composite score (e.g., the difference of differences (DoD) for a data object corresponding to an individual (i), the analytics server can calculate the following:

$${DoD}_{i} = {Score}_{i , {trt}_{A}} - {Score}_{i , {trt}_{B}} = ( {Score}_{i , b , {trt}_{A}} - {Score}_{i , r , {trt}_{A}} ) - ( {Score}_{i , b , {trt}_{B}} - {Score}_{i , r , {trt}_{B}} ) = ( {Score}_{i , b , {trt}_{A}} - {Score}_{i , b , {trt}_{B}} ) - ( {Score}_{i , r , {trt}_{A}} - {Score}_{i , r , {trt}_{B}} ) = {bD}_{i} - {rD}_{i}$$

Here, bDᵢ and rDᵢ correspond to the first event (e.g., benefit event) and second event (e.g., risk event) differences in the scores, respectively. Composite scores where DoDᵢ>0, DoDᵢ<0, and DoDᵢ=0 will be understood as signaling evidence of favorable, unfavorable, and null efficacy for treatment, respectively.

At operation 212, the analytics server can provide data associated with a user interface to cause the user interface to be displayed on a device, such as the client device described above. For example, the analytics server can provide data associated with a user interface that is configured to cause the user interface to indicate whether the first intervention satisfies a threshold value indicating that the clinical benefits of administering the first intervention outweigh the risks of administering the first intervention, in view of the clinical benefits and risks of other (e.g., the second) intervention. Satisfaction of the threshold value can mean that the analytics server has determined the composite score is a positive value when a similar score for the second intervention is subtracted from the composite score. In some examples, satisfaction of the threshold value can mean that the composite score is at least the same as, or greater than, a predetermined score (e.g., a positive value set by the clinician controlling the client device indicating a minimum score). The analytics server can include an element (for example, an icon, a label, or a visual marker) in the user interface that represents a comparison outcome between the composite score and the threshold value.

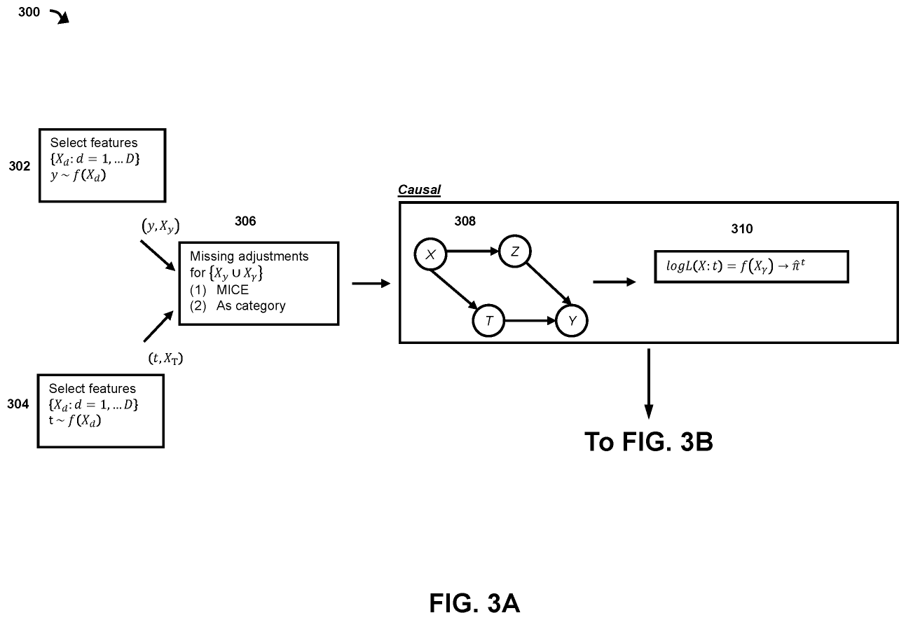

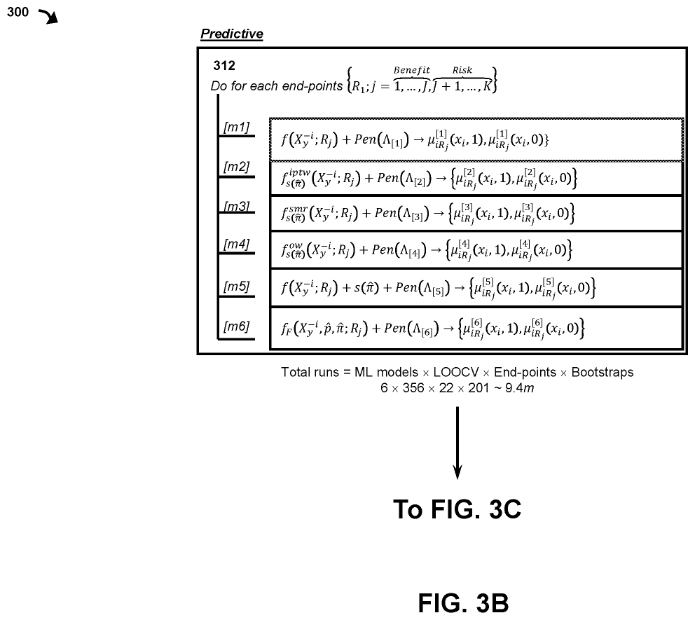

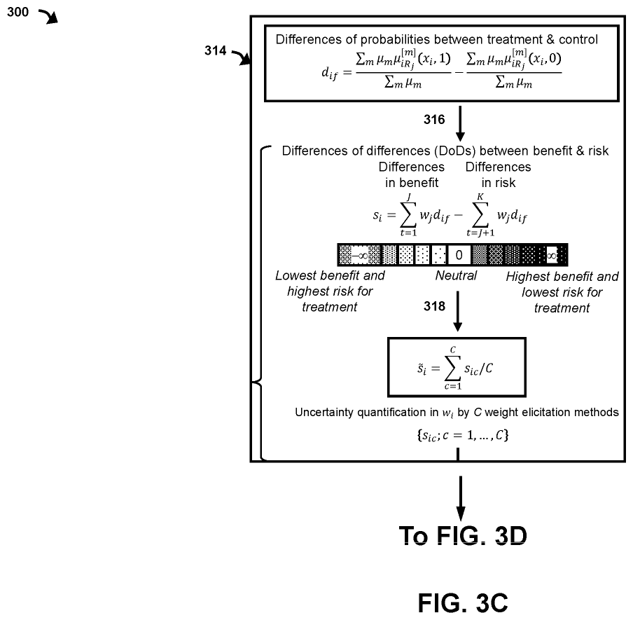

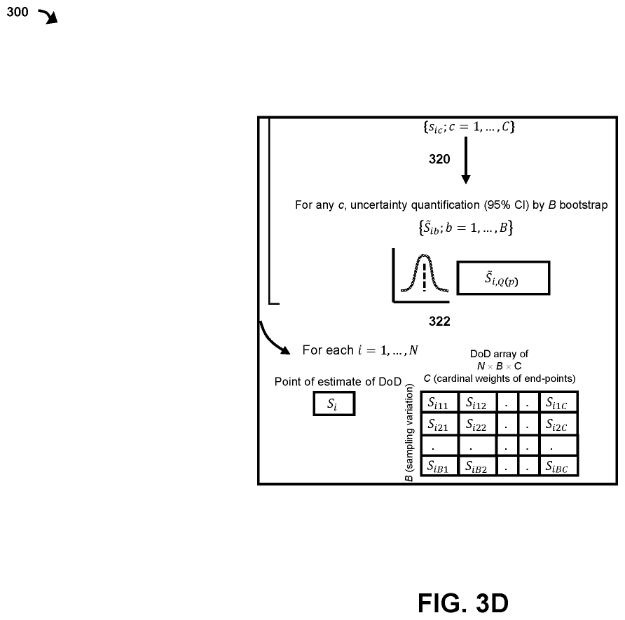

FIGS. 3A-3D are a diagram illustrating a process 300 for analyzing data objects using machine learning models described with respect to FIG. 2, in accordance with one or more embodiments described herein. In some implementations, one or more of the functions described with respect to the process 300 can be performed (e.g., completely, partially, and/or the like) by an analytics server that is the same as, or similar to, the analytics server 102 of FIG. 1. In some implementations, one or more of the functions described with respect to the process 300 can be performed (e.g., completely, partially, and/or the like) by another device or group of devices separate from and/or including the analytics server, such as by one or more client devices that are the same as, or similar to, the client device 126 of FIG. 1.

At operations 302 and 304, the analytics server can select one or more sets of features (represented as a set X_{d} of features) to use when training one or more machine learning models (illustrated as log L (X;t) with respect to training at operation 310 and as f(X) when used at inference at operation 312) as described herein. For example, the analytics server can execute a series of analyses (for example, Kaplan-Meier analyses) to screen and select an initial baseline set of features Xa that are associated with one or more events (referred to as time-to-event endpoints such as overall survival, event-free survival, relapse-free survival, response duration, or combinations thereof) for data objects representing patients treated with ven/aza and ven/aza/cusa. The analytics server can use any p-values greater than 0.30 obtained from testing the equality of survival curves for any of the 10 test statistics (evaluated with a battery of two-sided survival-curve tests: log-rank, Tarone-Ware, Fleming-Harrington weighted for early and for late follow-up, multi-directional weighted log-rank, Max-Combo, K-sample omnibus non-proportional hazards, and restricted mean survival time) to allow retention of corresponding features in a confounding set of features. In an example, the analytics server can refer to details of these test statistics and can omit redundant details from the present description. In some examples, the analytics server can screen out noise variables and select a sparser list of confounding variables to allow control in subsequent multivariate analyses. The analytics server can use standardized mean difference values greater than 0.20 to determine a set of features that are meaningfully different between ven/aza and ven/aza/cusa.

At operation 306, the analytics server can determine a union of these disparate feature sets {x_{y}∪x_{T}} generated at operations 302 and 304 to determine a final list of features for use in training the machine learning models. The analytics server can then use these feature sets to generate a second set of data objects for training (as described above). In examples, the analytics server can use a MICE algorithm (multiple imputation by chained equations) to process missing indicators in the second set of data objects to update the data objects and make them compatible with the model(s) being implemented. Additionally, or alternatively, the analytics server can process the data objects by treating the missing indicator values represented in data objects as categories (for example, assigning a distinct categorical value to missing data points) instead of applying an imputation algorithm. In examples, the analytics server can designate missing values as a discrete category to allow a clear identification of missing data during data preprocessing.

At operation 308, the analytics server can determine correlations (e.g., causal connections) between an initial set of features X, treatments T that can be administered, and a set of marker values Z representing the states of patients treated (as represented by one or more data objects as described herein), resulting in events Y (e.g., benefit events, risk events, etc.). These correlations are represented using a directed acrylic graph (DAG) as shown, where a patient X that receives a treatment T has a probability Y of being associated with one or more events Z.

To reduce the magnitude of systematic differences and selection bias in the data objects representing individuals that are treated with a therapy (ven/aza/cusa) and individuals that represent a control (ven/aza), the analytics server can sample propensity scores, signaling conditional probability of a given individual receiving a particularly therapy (e.g., ven/aza/cusa), that are estimated using four machine learning (ML) models: (a) multiple generalized linear model (GLM) with logit link function, (b) multiple generalized linear model with ridge (mGLMr) with logit link function, (c) random forest (RF), and (d) ensemble of the above three models (e.g., determined by averaging the outputs of combinations of models a-c). The analytics server can then select a final model from among these four machine learning models that provided the best numerical performance to compute the underlying sample propensity scores (PS) indicating the likelihood that a given data object corresponds to an individual that can be treated with a therapy. A DAG can then be used to determine the set of controlling variables to be adjusted for in a treatment model (age, prior history of comorbidities, platelet transfusions at treatment start date, red blood cells at a treatment start date, etc.) and outcome models (discussed below). Then analytics server can then determine estimated propensity scores to compute propensity weights, such as stabilized inverse probability of treatment weights (IPTWs), standardized mortality/morbidity ratio (SMR) weights, and overlap weights (OW). In some embodiments, the analytics server can then update the data objects by processing each data object using the selected model and associating the data object with an indication of whether the corresponding individual is eligible for the therapy.

As described above, different types of models (e.g., a multiple generalized linear model with ridge (mGLMr) or a multiple generalized linear model with a logit link function) can be implemented to support binary classification of the data objects as corresponding to individuals that satisfy conditions to be treated with a therapy. For example, the analytics server can configure the models to generate outputs indicating probabilities of individuals corresponding to data objects being eligible to receive a therapy by processing the data objects including multiple indicators (specifying indicator values/marker values). Additionally, or alternatively, attention-based models such as transformers or components thereof can be implemented. In some embodiments, the analytics server can receive inputs in the form of data objects that represent indicators corresponding to specific marker values, which are obtained by the analytics server from the client device, laboratory system, sequencing system, etc.). These indicators can be processed, normalized, and formatted by the analytics server implementing a data ingest pipeline before being provided as inputs to the model in a model development environment. In these examples, the analytics server can configure the model development environment to execute the models in accordance with the data objects to configure and/or cause the models to generate estimated probabilities that each individual satisfies the criteria for acceptance of a therapy.

In some embodiments, the analytics server can train the models using data objects corresponding to treatment profiles stored in a global patient database and/or refined dataset. These data objects can include feature vectors and be annotated with binary outputs that indicate whether corresponding individuals satisfy the acceptance criteria for therapy (described above). Each feature vector of a data object can then be processed through the models to generate a predicted probability, which is then compared with the actual outcome using a defined loss function such as negative log-likelihood. The weights of the models can be iteratively updated until the models converge (e.g., until the difference between the predicted probability and the actual probability satisfies (e.g., is less than) a predetermined threshold value).

Additionally, or alternatively, the analytics server can implement a random forest model employing an ensemble of decision trees to support binary classification of the data objects as corresponding to individuals that satisfy criteria for treatment with the therapy. In some embodiments, the analytics server can configure the random forest models to generate outputs indicating probabilities that individuals corresponding to the data objects are eligible to receive a therapy by processing the data objects including the multiple indicators. For example, the analytics server can receive the data objects that represent indicators corresponding to specific marker values obtained from client devices, laboratory systems, or sequencing systems and implement a random forest model to generate outputs for each data object indicating whether a corresponding individual is eligible to receive the therapy. In some embodiments, the analytics server can process the inputs where each data object comprises a feature vector containing indicator values. These feature vectors can then be used to execute the random forest model to generate estimated probabilities for each data object, where the output reflects an aggregated classification generated by a plurality of decision trees.

In some embodiments, the analytics server can train the random forest model using the data objects. For example, the analytics server can provide each data object to be processed by the multiple decision trees where each tree is constructed by splitting the indicator values represented by the data object based on indicator thresholds. The analytics server can then iteratively update the decision trees (e.g., the indicator thresholds) using bootstrap samples of the training data, and update decision criteria at each node until the aggregated predicted probability converges with the observed outcome as determined by a defined loss function.

At operation 312 the analytics server can configure and train one or more models (referred to above as “outcome models”) using the one or more data objects. For example, as shown, the analytics server can train six machine learning models (e.g., using leave-one-out-cross-validation (LOOCV) techniques described herein) to estimate subject-specific probabilities that one or more events will occur in response to administration of one or more therapies. Counterfactual estimates of observed events (in probability) for both the treatment and control arms can then be computed using S-learning or G-computation approaches. The counterfactual estimation of pseudo-outcomes can involve a fixed analytical patient cohort for both treatment arms, assuming that all individuals received ven/aza and ven/aza/cusa simultaneously, which allows for a balanced comparison while reducing the risk of spurious effects arising from imbalances in sample sizes between ven/aza and ven/aza/cusa. Weighted machine learning models, such as penalized multivariable Cox-proportional hazard models (mCOXr) and mGLMr with an L2-norm penalty, can be fitted along with propensity score weights (e.g., based on inverse probability of treatment weighting (IPTW), standard mortality ratios (SMR), and/or overlap weighting (OW)). In a separate additive model, the analytics server can adjust the logits of propensity scores in both penalized time-to-event and logistic regressions as a smooth function which can be approximated using piecewise constant splines. Finally, a tree-based approach, such as causal survival forests (CSF), can be implemented to counterfactually estimate pseudo-outcomes, in which all tuning parameters for both the treatment and outcome models are set at default. In some embodiments, the analytics server can implement an ensemble (e.g., meta-learner) synthesizing the estimates of all six ML models was used with equal weights to account for epistemic variations in the ML models. Alternatively, a weighted average can be used, with weights being the reciprocal of the corresponding likelihood estimates of uncertainty for each model. To quantify the uncertainty in estimates, the analytics server can implement fractional random weight bootstrap (FRWB) (e.g., 1000 bootstraps), and the resulting empirical distribution of bootstrap estimates used to obtain percentile-based 95% confidence intervals. The set of controlling variables to partial out the underlying effects of mediators and confounders was determined via clinical jurisdiction and DAG. To circumvent creating over-saturated models, the composite risk variables such as the refined risk model (RRM) and European LeukemiaNet (ELN22) can be used instead of adjusting each genetics and phenotypic features in the models.

At operation 312, the analytics server can execute the six models (configured at operation 310) in accordance with a data object (e.g., a later-obtained data object) representing an individual to generate six net scores for the individual. In some examples, the analytics server can process the data object (for example, representing portions of a treatment profile or a limited treatment profile) to extract features that can be used as inputs for each of the six models. The analytics server can independently execute each model to compute net scores that can represent the outcome of a corresponding intervention (e.g., the probability that one or more benefit events will occur, and one or more risk events will occur). In examples, the analytics server can then execute each model to obtain outputs from each model. In this example, the analytics server can compile the six net scores for subsequent evaluation of the individual across multiple interventions. In some examples, the analytics server can forward the compiled net scores to allow further analytics processes or decision-making steps based on the evaluated outcomes.

At operation 314, the analytics server can determine differences of probabilities (referred to above as a difference of differences or d_{if}) between administration of a therapy (e.g., a given treatment) and non-administration of the therapy (e.g., a control) based on the output of each model. For example, with respect to a given model m, the analytics server can compute the difference of differences for an individual (for example, DoDᵢ as described above) by subtracting a score for treatment B (Score_{i,trt}_{_{B}}) from a score for treatment A (Score_{i,trt}_{_{A}}), where the score for treatment A can be derived by subtracting a second score indicating the probability that a risk event will occur (for example, Score_{ir,trt}_{_{A}}) from a first score indicating the probability that a benefit event will occur (for example, Score_{i,b,trt}_{_{A}}) and similarly for treatment B. In some examples, the analytics server can express the calculation as the difference between the difference of benefit scores and risk scores for treatments A and B. In this example, the analytics server can determine the difference of differences to allow for a comprehensive evaluation of the changes between benefits events that may occur and risk events that may occur measures for both treatment types.

At operation 316, the analytics server can aggregate each net score to determine a composite score derived from the outputs of each of the models. For example, the analytics server can collect the net scores generated based on the analytics server executing the individual models and apply an arithmetic or statistical method (for example, a summation or a weighted average) to form a composite score. In an example, the analytics server can then use the aggregated composite score to represent an overall evaluation metric that can be compared across interventions or used for further analysis. This evaluation metric can be represented between a range (e.g., from −X to +X) where lower values represent proportionally lower probabilities of achieving a desired benefit event while avoiding an undesired risk event.

At operation 318, the analytics server can update the outputs of the models (e.g., the probabilities that various events will occur) based on weights assigned to each type of event (e.g., endpoint) by a clinician corresponding to a particular model output. For example, the analytics server can apply weight elicitation methods (for example, preference or ranking techniques) to determine a weight that corresponds to a parameter or feature within a composite score. In examples, uncertainty quantification can allow estimation of the degree of confidence or variability associated with the elicited weight so that the overall contribution of each model or feature can be interpreted in light of the potential variability.

At operation 320, the analytics server can compute a 95 percent confidence interval for a parameter represented by C (representing a number of users that are directly or indirectly operating the analytics server to assign weights as described herein) using a bootstrap method with B iterations. In some examples, the analytics server can repeat resampling (for example, selecting samples with replacement) B times to compute distributions of estimates for C, where the results can be used to derive lower and upper bounds of a confidence interval.

At operation 322, the analytics server can determine a set of point estimates (si) for a plurality of combinations of events (e.g., combinations of risk events and benefit events). For example, the analytics server can determine a set of point estimates (si) that can represent a series of probabilities corresponding to different pairs of events, such as benefit events and risk events. In some examples, the analytics server can compute individual probability values for each combination of a benefit event and a risk event by processing the data objects as described above and applying statistical models that can measure the probability of each event pair occurring. In an example, each computed point estimate can serve as an indication (for example, a predictive metric or outcome variable) of the degree to which a specific pairing of benefit and risk events is realized, thereby allowing an overall probabilistic assessment across the range of combinations. In some examples, the analytics server can aggregate these point estimates to form a matrix of probabilities that can be used to inform decision-making processes or to provide further insights into the interplay between benefit and risk events.

In some embodiments, the analytics server can use the point estimates to generate a user interface at a client device controlled by a clinician. In some examples, the analytics server can aggregate the computed probabilities corresponding to different event pairs and generate display data configured to cause a display device of the client device to output the values for visualization (for example, as single value, a chart, a table, etc.) reflecting the balance between benefits and risks for the therapy on a per-patient basis. In some examples, the user interface can indicate whether the probability values suggest that the benefits outweigh the risks by presenting an overall metric or threshold indicator that can guide the clinician's decision-making process. In this example, the interface can allow a clinician to determine, based on the point estimates and computed metrics, whether a therapy should be administered to the corresponding individual by providing indications of whether a therapy is likely to result in net positive outcomes.

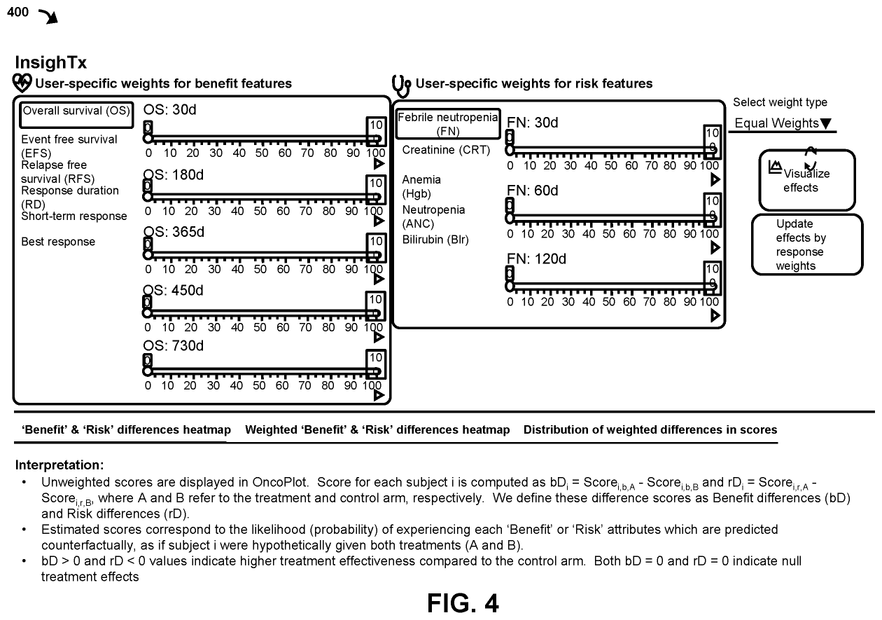

FIG. 4 is a diagram illustrating an interactive user interface 400 that can be implemented, in accordance with one or more embodiments described herein. In some implementations, one or more of the functions described with respect to the user interface 400 can be performed (e.g., completely, partially, and/or the like) by an analytics server that is the same as, or similar to, the analytics server 102 of FIG. 1. In some implementations, one or more of the functions described with respect to the user interface 400 can be performed (e.g., completely, partially, and/or the like) by another device or group of devices separate from and/or including the analytics server, such as by one or more client devices that are the same as, or similar to, the client device 126 of FIG. 1.

The user interface 400 can present a first region containing user-specific weights for benefit features and a second region containing user-specific weights for risk features. For example, the first region can include selectable benefit feature types, such as overall survival (OS), event-free survival (EFS), relapse-free survival (RFS), response duration (RD), short-term response, and/or best response, each associated with one or more predetermined time intervals (e.g., 30 days, 180 days, 365 days, 450 days, and/or 730 days). The second region can include selectable risk feature types, such as febrile neutropenia (FN), creatinine (CRT), anemia (Hgb), neutropenia (ANC), and/or bilirubin (Blr), each associated with one or more predetermined time intervals (e.g., 30 days, 60 days, and/or 120 days). In some implementations, the user interface 400 can present an adjustable input element (e.g., a slider, a numeric field, etc.) adjacent to each benefit feature type and each risk feature type at each time interval, the adjustable input element allowing a clinician to assign a weight value within a predefined range (e.g., 0 to 100) to the corresponding feature type.

The user interface 400 can include a weight type selector that allows the clinician to select a weighting scheme to be applied across the benefit feature types and the risk feature types. For example, the weight type selector can include an option to apply equal weights across all feature types and time intervals, or to apply a custom weighting scheme in which the clinician assigns individual weight values to each feature type. In some implementations, the user interface 400 can include a visualization control element (e.g., a button labeled to indicate visualization of effects) that, upon selection, causes the analytics server to generate and display one or more output visualizations reflecting differences in benefit scores and risk scores computed based on the assigned weight values. The output visualizations can include a benefit and risk differences heatmap, a weighted benefit and risk differences heatmap, and/or a distribution of weighted differences in scores. In some implementations, the user interface 400 can include an update control element that, upon selection, causes the analytics server to recalculate the output visualizations in response to changes to one or more weight values assigned by the clinician.

The user interface 400 can include an interpretive text region that provides descriptions of the scores displayed in the output visualizations. For example, the interpretive text region can indicate that unweighted scores correspond to a score computed for each individual by subtracting a score for a control arm from a score for a treatment arm, and that the resulting difference scores can be categorized as benefit differences (bD) and risk differences (rD). In some implementations, the interpretive text region can indicate that estimated scores correspond to probabilities of experiencing each benefit feature type or each risk feature type, where the probabilities are predicted counterfactually under both a treatment arm and a control arm for a given individual. The interpretive text region can further indicate that a positive benefit difference value and a negative risk difference value signal higher treatment effectiveness of the treatment arm relative to the control arm, and that a benefit difference value of zero and a risk difference value of zero signal a null treatment effect.

In some embodiments, the analytics server can generate a user interface 400 at a client device controlled by a clinician. For example, the user interface 400 can present a visualization (for example, a chart, a table, etc., with corresponding sliders or other input symbols) that reflects desired weights between first events (e.g., benefit events) and second events (e.g., risk events) for a specific therapy. In some examples, the user interface can include interactive controls that allow the clinician to select one or more event types for the first events, such as overall survival, event-free survival, relapse-free survival, response duration, short-term response, and/or best response at specific time intervals (for example, 30 days, 180 days, 365 days, 450 days, and 730 days). Similarly, the user interface can include interactive controls that allow the clinician to select one or more event types for the second events, such as febrile neutropenia, creatinine, anemia, neutropenia, and/or bilirubin at specific time intervals (for example, 30 days, 180 days, 365 days, 450 days, and 730 days). In this example, the clinician can also input weight values corresponding to each selected endpoint type to be used when evaluating additional data objects that represent indicators corresponding to individuals being treated for a given disease. In some examples, the analytics server can incorporate these clinician-defined endpoint types and weight values when configuring one or more models as described herein. Once configured, the analytics server can then use the models as described above to generate indications of whether providing a particular therapy will result in a net benefit in accordance with the weights configured by the clinician.

FIG. 5 is a flow chart illustrating a process 500 for reducing secured data objects by removing indicator fields that fail a threshold criterion, encoding remaining indicator values into fixed format feature vectors, training a plurality of machine learning models, excluding models that fail a prediction-error threshold, generating intervention scores, determining a composite intervention score, and modifying an intervention-selection interface at a remote client device, in accordance with one or more embodiments described herein. In some implementations, one or more of the functions described with respect to the process 500 can be performed (e.g., completely, partially, and/or the like) by an analytics server that is the same as, or similar to, the analytics server 102 of FIG. 1. In some implementations, one or more of the functions described with respect to the process 500 can be performed (e.g., completely, partially, and/or the like) by another device or group of devices separate from and/or including the analytics server, such as by one or more client devices that are the same as, or similar to, the client device 126 of FIG. 1. FIG. 5 illustrates a variation of the method described with respect to FIG. 2, in which the process 500 provides additional detail regarding data reduction, model qualification, and interface modification operations.

At operation 502, the analytics server can obtain a set of secured data objects for individuals being treated for a condition. For example, each secured data object can include a tokenized subject identifier and a plurality of indicator fields storing corresponding indicator values. The tokenized subject identifier can represent a de-identified reference to the individual, and the indicator fields can store quantitative measurements (e.g., biomarker values, laboratory results, demographic data, treatment parameters, and/or the like) associated with the condition and the treatment of the individual. In some examples, the analytics server can obtain the secured data objects from one or more data sources, such as a global patient database, a refined dataset, or a combination thereof, as described with respect to FIG. 1. The analytics server can identify the secured data objects from among a larger plurality of data objects by determining that the secured data objects match one or more preestablished conditions (e.g., that the individuals are diagnosed with a condition such as AML, that the individuals have been administered one or more therapies, and/or the like).

At operation 504, the analytics server can perform an association value determination for each indicator field of each secured data object. For example, the analytics server can determine, for each indicator field, an association value representing a correlation of the indicator field with the condition or with an intervention outcome. In some examples, the association value can be a p-value computed based on one or more statistical tests (e.g., Kaplan-Meier analyses, log-rank tests, weighted log-rank tests, and/or other survival-curve equality tests as described with respect to FIG. 3A). The association value can represent a degree of statistical association between the indicator type corresponding to the indicator field and the condition being analyzed or the outcome of an intervention administered to treat the condition. In some examples, the analytics server can additionally or alternatively compute standardized mean difference values to identify indicator fields that exhibit meaningful differences between intervention groups.

At operation 506, the analytics server can perform indicator field removal by removing, from each secured data object, indicator fields having indicator values that fail a threshold criterion. For example, the analytics server can compare each association value determined at operation 504 to a threshold value (e.g., a threshold p-value of 0.3 or greater, or another threshold value configured by a clinician or set by default). In response to determining that an association value for a given indicator field does not satisfy the threshold criterion, the analytics server can remove the corresponding indicator field from the secured data object. By removing indicator fields that do not meet the threshold criterion, the analytics server can reduce the dimensionality of the data objects and remove features that are not sufficiently correlated with the condition or intervention outcome, thereby reducing the amount of data that is processed in subsequent operations.

At operation 508, the analytics server can perform feature vector encoding by encoding the remaining indicator values into a fixed format feature vector having fewer fields than the plurality of indicator fields of the original secured data object. For example, after the indicator fields that fail the threshold criterion have been removed at operation 506, the analytics server can encode the remaining indicator values into a fixed format feature vector. The fixed format feature vector can represent a standardized data structure in which each remaining indicator value is mapped to a corresponding position, and the total number of fields in the feature vector is less than the total number of indicator fields in the original secured data object. In some examples, the encoding can include normalizing the indicator values, converting categorical indicator values into numerical representations, and/or applying one or more imputation techniques (e.g., a MICE algorithm or categorical assignment of missing values) to address missing indicator values, as described with respect to FIGS. 3A-3D.

At operation 510, the analytics server can store the fixed format feature vector in a reduced secured data object that excludes direct patient identifiers. For example, the analytics server can generate the reduced secured data object such that the reduced secured data object includes the fixed format feature vector generated at operation 508 while omitting any direct patient identifiers (e.g., names, addresses, social security numbers, and/or the like) that may have been present in the original secured data object. The reduced secured data object can retain the tokenized subject identifier to allow association of the reduced secured data object with the corresponding individual without exposing personally identifiable information. By excluding direct patient identifiers and retaining a reduced set of indicator values, the reduced secured data object can reduce the risk of unauthorized reconstruction of sensitive patient data in the event of a security breach.

At operation 512, the analytics server can perform transmission to model-training pipeline by transmitting the reduced secured data objects, and not the original secured data objects, to a model-training pipeline. For example, the analytics server can transmit the reduced secured data objects generated at operation 510 to a model-training pipeline that can reside on the same analytics server or on a separate computing environment (e.g., a distributed or cloud-based training environment). By transmitting the reduced secured data objects instead of the original secured data objects, the analytics server can reduce the network transfer size and the memory usage allocated for model training. The reduction in data volume can decrease latency during transmission and conserve bandwidth, which can be advantageous in distributed or cloud-based environments where large volumes of data are exchanged during training operations.

At operation 514, the analytics server can perform model training by training, using the reduced secured data objects and a cross-validation schedule, a plurality of machine learning models having different respective architectures. For example, the analytics server can train the plurality of machine learning models to generate, for a given reduced secured data object and an intervention identifier, a benefit-event probability and an adverse-event probability within a predetermined time interval. In some examples, the different architectures can include convolutional neural networks, multilayer perceptrons, decision tree ensembles, penalized regression models, causal survival forests, and/or the like, as described with respect to FIGS. 2 and 3A-3D. The cross-validation schedule can include a leave-one-out cross-validation (LOOCV) technique or another partitioning strategy in which subsets of reduced secured data objects are iteratively reserved for evaluation while the remaining reduced secured data objects are used for training. The analytics server can iteratively update the parameters of each machine learning model until the benefit-event probabilities and adverse-event probabilities generated by the model converge with known outcomes for the reduced secured data objects in the training set.

At operation 516, the analytics server can perform model exclusion by excluding from subsequent scoring any machine learning model having a cross-validation prediction error greater than a prediction-error threshold. For example, after training the plurality of machine learning models at operation 514, the analytics server can evaluate each trained model by computing prediction errors based on the cross-validation results. The analytics server can compare the prediction error of each model to the prediction-error threshold and, in response to determining that the prediction error of a given model exceeds the prediction-error threshold, exclude the given model from subsequent scoring operations. By excluding underperforming models, the analytics server can reduce the likelihood that inaccurate predictions influence the intervention scores and composite intervention score determined in subsequent operations, thereby improving the reliability of the outputs.

At operation 518, the analytics server can perform model execution by executing the remaining machine learning models (e.g., the models not excluded at operation 516) for at least one later-obtained reduced secured data object. For example, the analytics server can receive a later-obtained reduced secured data object corresponding to an individual for whom an intervention recommendation is being evaluated. The analytics server can execute each remaining machine learning model using a first intervention identifier (e.g., an identifier corresponding to administration of a first therapy such as ven/aza/cusa) and a second intervention identifier (e.g., an identifier corresponding to administration of a second therapy such as ven/aza or non-administration of any agent) to generate a first intervention score and a second intervention score. The first intervention score and the second intervention score can each be derived from corresponding benefit-event probabilities and adverse-event probabilities generated by the remaining machine learning models when processing the fixed format feature vector of the later-obtained reduced secured data object.

At operation 520, the analytics server can perform composite intervention score determination based on a difference between the first intervention score and the second intervention score generated at operation 518. For example, the analytics server can compute the composite intervention score as a difference of differences, as described with respect to FIG. 2, where the composite intervention score represents the relative advantage of the first intervention over the second intervention when accounting for both benefit-event probabilities and adverse-event probabilities. A positive composite intervention score can indicate that the first intervention is associated with a favorable balance of benefits relative to risks when compared to the second intervention. A negative composite intervention score can indicate that the first intervention is associated with an unfavorable balance. A composite intervention score of approximately zero can indicate a null treatment effect between the first intervention and the second intervention.

At operation 522, the analytics server can perform control record generation in response to determining that the composite intervention score satisfies a threshold. For example, the analytics server can compare the composite intervention score to a predetermined threshold value and, in response to determining that the composite intervention score satisfies the threshold, automatically generate a control record for a remote client device. The control record can be configured to modify an intervention-selection interface at the remote client device by enabling presentation of the first intervention and suppressing or deprioritizing presentation of the second intervention. In some examples, the control record can include interface modification data (e.g., display instructions, element visibility flags, ranking values, and/or the like) that, when received and processed by the remote client device, cause the intervention-selection interface to visually distinguish the first intervention from the second intervention. For instance, the interface can display the first intervention with a visual indicator signaling suitability (e.g., a highlighted label, a ranking position, an enabled selection element, and/or the like) while suppressing or deprioritizing the second intervention (e.g., graying out a corresponding selection element, repositioning the second intervention lower in a list, removing the second intervention from a default view, and/or the like). In this way, the analytics server can provide clinicians with actionable intervention recommendations that are derived from the composite intervention score and presented through the modified intervention-selection interface at the remote client device.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Some embodiments of the present disclosure are described herein in connection with a threshold. As described herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for training machine learning systems to classify secured data objects comprising sets of indicators based on predefined features, the system comprising:
one or more processors; and
non-transitory memory storing instructions that, when executed by the one or more processors, cause the system to:
obtain a set of secured data objects for individuals being treated for a condition, each secured data object including a tokenized subject identifier and a plurality of indicator fields storing corresponding indicator values;
for each secured data object:
determine, for each indicator field, an association value representing correlation of the indicator field with the condition or with an intervention outcome;
remove from the secured data object indicator fields having indicator values that fail a threshold criterion;
encode remaining indicator values into a fixed format feature vector having fewer fields than the plurality of indicator fields; and
store the fixed format feature vector in a reduced secured data object that excludes direct patient identifiers;
transmit only the reduced secured data objects, and not the secured data objects, to a model-training pipeline, thereby reducing network transfer size and memory usage for model training;
train, using the reduced secured data objects and a cross-validation schedule, a plurality of machine learning models having different respective architectures to generate, for a reduced secured data object and an intervention identifier, a benefit-event probability and an adverse-event probability within a predetermined time interval;
exclude from subsequent scoring any machine learning model having cross-validation prediction error greater than a prediction-error threshold;
execute remaining machine learning models for at least one later-obtained reduced secured data object using a first intervention identifier and a second intervention identifier to generate a first intervention score and a second intervention score from corresponding benefit-event probabilities and adverse-event probabilities;
determine a composite intervention score based on a difference between the first intervention score and the second intervention score; and
in response to determining that the composite intervention score satisfies a threshold, automatically generate a control record for a remote client device to modify an intervention-selection interface by enabling presentation of the first intervention and suppressing or deprioritizing presentation of the second intervention.

2. The system of claim 1, wherein the instructions further cause the system to obtain the set of secured data objects by:
determining one or more secured data objects that match one or more preestablished conditions from among a plurality of secured data objects; and
generating the set of secured data objects based on the one or more secured data objects that match the one or more preestablished conditions.

3. The system of claim 1, wherein the instructions further cause the system to generate the reduced secured data objects by:
determining p-values for each indicator field corresponding to each indicator value from the plurality of indicator fields, the p-values representing a degree of association between each indicator field and the condition;
in response to comparing each p-value to a threshold p-value, determining the indicator fields having indicator values that satisfy the threshold p-value; and
encoding the indicator values of the indicator fields that satisfy the threshold p-value into the fixed format feature vector of each reduced secured data object.

4. The system of claim 3, wherein the threshold p-value is 0.3 or greater.

5. The system of claim 1, wherein the instructions that cause the system to train the plurality of machine learning models using the reduced secured data objects further cause the system to:
for each reduced secured data object, iteratively provide at least a portion of the fixed format feature vector as input to each machine learning model of the plurality of machine learning models until the benefit-event probability and the adverse-event probability satisfy a threshold difference when compared to a known benefit-event outcome and a known adverse-event outcome for each reduced secured data object.

6. The system of claim 5, wherein the instructions further cause the system to:
remove a subset of reduced secured data objects from among the reduced secured data objects;
in response to removing the subset of reduced secured data objects, train the plurality of machine learning models; and
execute the plurality of machine learning models in accordance with the subset of reduced secured data objects to determine a set of prediction errors.

7. The system of claim 6, wherein the subset of reduced secured data objects comprises a first subset of reduced secured data objects, and
wherein the instructions further cause the system to:
determine that a prediction error of at least one machine learning model of the plurality of machine learning models satisfies the prediction-error threshold;
remove a second subset of reduced secured data objects from among the reduced secured data objects;
in response to removing the second subset of reduced secured data objects, train the plurality of machine learning models; and
execute the plurality of machine learning models in accordance with the second subset of reduced secured data objects to determine a set of prediction errors.

8. The system of claim 1, wherein the instructions that cause the system to execute the remaining machine learning models further cause the system to:
execute each remaining machine learning model based on the fixed format feature vector of the at least one later-obtained reduced secured data object using the first intervention identifier to generate the first intervention score and using the second intervention identifier to generate the second intervention score.

9. The system of claim 8, wherein the instructions that cause the system to determine the composite intervention score further cause the system to:
determine the composite intervention score for the at least one later-obtained reduced secured data object based on a difference between benefit-event probabilities generated using the first intervention identifier and benefit-event probabilities generated using the second intervention identifier, and a difference between adverse-event probabilities generated using the first intervention identifier and adverse-event probabilities generated using the second intervention identifier, the benefit-event probabilities and adverse-event probabilities generated by executing each remaining machine learning model based on the fixed format feature vector of the at least one later-obtained reduced secured data object.

10. A method comprising:
obtaining a set of secured data objects for individuals being treated for a condition, each secured data object comprising a set of indicator values;
generating a set of second secured data objects for the set of secured data objects, each second secured data object corresponding to a secured data object and comprising a subset of indicator values from the set of indicator values;
training a set of machine learning systems based on the set of second secured data objects to generate outputs comprising first outputs indicating that a first event will occur and second outputs indicating that a second event will occur within a predetermined amount of time after a condition is satisfied, each machine learning system of the set of machine learning systems comprising a different architecture;
generating a first net score for a first intervention and a second net score for a second intervention based on the set of machine learning systems and at least one second secured data object;
determining a composite score for the at least one second secured data object based on the first net score the second net score; and
providing data associated with a user interface to cause the user interface to be displayed on a device, the user interface indicating whether the first intervention satisfies a threshold value indicating suitability of the first intervention based on the composite score for the at least one second secured data object.

11. The method of claim 10, wherein obtaining the set of secured data objects comprises:
determining one or more secured data objects that match one or more preestablished conditions from among a plurality of secured data objects; and
generating the set of secured data objects on the one or more secured data objects that match the one or more preestablished conditions.

12. The method of claim 10, wherein generating the set of second secured data objects comprises:
determining p-values for each indicator type corresponding to each indicator value from the set of indicator values, the p-values representing a degree of association between each indicator type and a condition;
in response to comparing each p-value to a threshold p-value, determining the subset of indicator values based on whether each p-value satisfies the threshold p-value; and
generating the set of second secured data objects based on the subset of indicator values.

13. The method of claim 12, wherein the threshold p-value is 0.3 or greater.

14. The method of claim 10, wherein configuring the set of machine learning systems based on the set of second secured data objects comprises:
for each secured data object of the set of second secured data objects, iteratively providing at least a portion of the subset of indicator values as input to each machine learning system of the set of machine learning systems until the first output indicating that the first event will occur and the second output indicating that the second event will occur satisfy a threshold difference when compared to a known first output indicating and a known second output indicating for each secured data object.

15. The method of claim 14, wherein configuring the set of machine learning systems comprises:
removing a subset of second secured data objects from among the set of second secured data objects;
in response to removing the subset of secured data objects, configuring the set of machine learning systems; and
executing the set of machine learning systems in accordance with the subset of second secured data objects to determine a set of prediction errors.

16. The method of claim 15, wherein the subset of second secured data objects comprises a first subset of secured data objects, and
wherein the method further comprises:
determining the prediction error of at least one machine learning system of the set of machine learning systems satisfies a prediction threshold;
removing a second subset of second secured data objects from among the set of second secured data objects;
in response to removing the second subset of secured data objects, configuring the set of machine learning systems; and
executing the set of machine learning systems in accordance with the second subset of second secured data objects to determine a set of prediction errors.

17. The method of claim 10, wherein generating the first net score for the first intervention and the second net score for the second intervention based on the set of machine learning systems and the at least one secured data object comprises:
executing each machine learning system of the set of machine learning systems based on the subset of indicator values of the at least one second secured data object to the first net score for the first intervention and the second net score for the second intervention.

18. The method of claim 17, wherein determining the composite score comprises:
determining the composite score for the at least one second secured data object based on a difference between third outputs indicating that the first event will occur and fourth outputs indicating that the second event will occur, the third outputs indicating and fourth outputs generated by executing each machine learning system of the set of machine learning systems based on the subset of indicator values of the at least one second secured data object.

19. One or more non-transitory computer-readable mediums storing instructions thereon that, when executed by one or more processors, cause the one or more processors to:
obtain a set of secured data objects for individuals being treated for a condition, each secured data object comprising a set of indicator values;
generate a set of second secured data objects for the set of secured data objects, each second secured data object corresponding to a secured data object and comprising a subset of indicator values from the set of indicator values;
train a set of machine learning systems based on the set of second secured data objects to generate outputs comprising first outputs indicating that a first event will occur and second outputs indicating that a second event will occur within a predetermined amount of time after a condition is satisfied, each machine learning system of the set of machine learning systems comprising a different architecture;
generate a first net score for a first intervention and a second net score for a second intervention based on the set of machine learning systems and at least one second secured data object;
determine a composite score for the at least one second secured data object based on the first net score the second net score; and
provide data associated with a user interface to cause the user interface to be displayed on a device, the user interface indicating whether the first intervention satisfies a threshold value indicating suitability of the first intervention based on the composite score for the at least one second secured data object.

20. The one or more non-transitory computer-readable mediums of claim 19, wherein the instructions that cause the one or more processors to obtain the set of secured data objects cause the one or more processors to:
determine one or more secured data objects that match one or more preestablished conditions from among a plurality of secured data objects; and
generate the set of secured data objects on the one or more secured data objects that match the one or more preestablished conditions.
